(19) United States
(12) Patent Application Publication
(10) Pub. No.: US 2024/0102051 A1
(43) Pub. Date: Mar. 28, 2024

(54) MULTIFUNCTIONAL COPOLYMERIC NANOPARTICLE

(71) Applicants: Seyyed Emad Hooshmand, Tehran (IR); Mahdi Karimi, Tehran (IR); Amir Hossein Vahabi, Tehran (IR)

(72) Inventors: Seyyed Emad Hooshmand, Tehran (IR); Mahdi Karimi, Tehran (IR); Amir Hossein Vahabi, Tehran (IR)

(73) Assignees: Seyyed Emad Hooshmand, Tehran, OT (IR); Mahdi Karimi, Tehran, OT (IR); Amir Hossein Vahabi, Tehran, OT (IR); Iran University of Medical Sciences, (omitted)

(21) Appl. No.: 18/513,783

(22) Filed: Nov. 20, 2023

Related U.S. Application Data
(63) Continuation-in-part of application No. PCT/IB2022/051356, filed on Feb. 16, 2022.
(60) Provisional application No. 63/192,117, filed on May 24, 2021.

(51) Int. Cl. C12N 15/87; A61K 9/51; A61K 31/795; A61P 31/04

(52) U.S. Cl. CPC C12N 15/87; A61K 9/5146; A61K 31/795; A61P 31/04

(57) ABSTRACT

A multifunctional copolymeric nanoparticle, including a plurality of 1,4-diazabicyclo[2.2.2]octane (DABCO) monomers crosslinked with a plurality of bromoacetyl cystamine (BBAC) monomers. Also, each of the plurality of DABCO monomers and each of the plurality of BBAC monomers alternate in sequence. the multifunctional copolymeric nanoparticle has a formula:

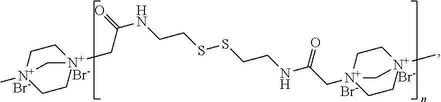

wherein n is an integer between 5 and 40.

### CROSS-REFERENCE TO RELATED APPLICATION

This application is a continuation-in-part of International Patent Application PCT/IB2022/051356, filed on Feb. 16, 2022, and entitled “MULTIFUNCTIONAL COPOLYMERIC NANOPARTICLE,” which takes priority from U.S. Provisional Patent Application Ser. No. 63/192,117, filed on May 24, 2021, and entitled “SYNTHESIS OF CATIONIC POLYMERS FOR GENE/CRISPR DELIVERY AND ANTIBACTERIAL APPLICATIONS,” which is incorporated herein by reference in its entirety.

### TECHNICAL FIELD

The present disclosure generally relates to nanoparticles, particularly to multifunctional copolymeric nanoparticles, and more particularly to synthesizing cationic multifunctional copolymeric nanoparticles to deliver nucleic acid to cells.

### BACKGROUND

One of the treatments for incurable diseases such as cancer is gene delivery, in which the genetic disorder is treated, and a normal and healthy gene replaces a defective gene by delivering a healthy gene and genetic material to cells. However, the main limitations of this method are the difficulty in transporting large molecules and the vulnerability of nucleic acids in the path outside the cell and inside the cells to reach the cell nucleus, which jeopardizes the biosafety aspects of using this method. The key to success in gene therapy is to develop efficient bio-safe carriers to transfer the gene to the target cells effectively without being destroyed by nucleases.

Many viral and non-viral vectors have been introduced as carriers to improve gene delivery. Viral vectors were initially the most widely used carriers in gene therapy due to their high transmission efficiency, but since viral carriers cause inflammation and stimulate the immune system, they are now not considered desirable options. Though non-viral vectors are less efficient at gene transfer than viral vectors, they are less toxic and not stimulate the immune system. Therefore, in recent years, extensive efforts have been made by various research groups to develop efficient and intelligent non-viral carriers due to the essential characteristics of non-viral carriers compared to viral carriers. Intelligent carriers are the carrier that reacts to an environmental factor, such as temperature, light, acidic environment, or reducing environment inside or outside the cell, and leave the drug or gene at the desired location.

Across the world, many diseases are generally caused by bacteria, including a wide variety of species. In the last decades, bacteria have acquired antibiotic resistance in the market, making infections triggered by drug-resistant bacteria hard to treat. However, some of the present antibacterial agents have merely affected a few bacteria or have not shown any antibacterial effect against certain bacteria. As a result, it is essential to develop antibacterial agents that selectively act on bacterial cells over mammalian cells. As extremely promising approaches to combating bacteria, nanomaterial-based antibacterial agents have attracted attention in recent years.

Hence, there is a need for a multi-purpose, safe, and efficient multifunctional nanoparticle that can be used for gene and drug delivery and antibacterial purposes. Also, there is a need for an intelligent and biocompatible nanoparticle as a non-viral vector with the same gene expression as viral vectors. Moreover, there is a need for antibacterial nanoparticles which have low toxicity on mammalian cells and show excellent antibacterial activity. Furthermore, there is a need for an efficient and simple method for synthesizing multifunctional nanoparticles that can be used for gene and drug delivery and antibacterial purposes.

### SUMMARY

This summary is intended to provide an overview of the subject matter of the present disclosure and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary multifunctional copolymeric nanoparticle. Exemplary multifunctional copolymeric nanoparticles may include a plurality of 1,4-diazabicyclo[2.2.2]octane (DABCO) monomers crosslinked with a plurality of bromoacetyl cystamine (BBAC) monomers. In an exemplary embodiment, each of the plurality of DABCO monomers and each of the plurality of BBAC monomers may alternate in sequence.

In an exemplary embodiment, the multifunctional copolymeric nanoparticle may have a formula:

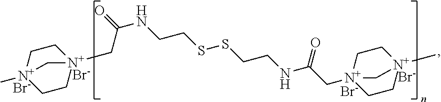

wherein n is an integer between 5 and 40. In an exemplary embodiment, the multifunctional copolymeric nanoparticle may be a cationic multifunctional copolymeric nanoparticle. In an exemplary embodiment, the multifunctional copolymeric nanoparticle may have a functional group, including an ammonium group, an amide group, and a disulfide group. In an exemplary embodiment, the multifunctional copolymeric nanoparticle may have a particle size between about 1 nm and about 2 μm.

In an exemplary embodiment, the multifunctional copolymeric nanoparticle may have antibacterial activity and anti-biofilm activity. In an exemplary embodiment, the multifunctional copolymeric nanoparticle may have antibacterial activity against gram-positive and gram-negative bacteria. In an exemplary embodiment, the gram-positive bacteria may include Staphylococcus aureus, Enterococcus. In an exemplary embodiment, the gram-negative bacteria may include Pseudomonas aeruginosa and Escherichia coli.

In an exemplary embodiment, the multifunctional copolymeric nanoparticle may be a redox-sensitive copolymeric nanoparticle. In an exemplary embodiment, the multifunctional copolymeric nanoparticle may be a transfection reagent for delivering a nucleic acid molecule into cells. In an exemplary embodiment, the nucleic acid molecule may include at least one of deoxyribonucleic acid (DNA) and ribonucleic acid (RNA). In an exemplary embodiment, the nucleic acid may include at least one of a plasmid, siRNA, miRNA, and mRNA. In an exemplary embodiment, a ratio of the nucleic acid molecule to the multifunctional copolymeric nanoparticle may be between about 1:1 and about 1:200.

In another general aspect, the present disclosure describes an exemplary method for synthesizing a multifunctional copolymeric nanoparticle. Exemplary method may include forming a mixture by mixing bromoacetyl cystamine (BBAC) and 1,4-diazabicyclo[2.2.2]octane (DABCO) with an alcoholic solvent, forming a precipitate by removing the alcoholic solvent from the mixture, and obtaining a purified multifunctional copolymeric nanoparticle by purifying the precipitate through washing the precipitate with an organic solvent.

In an exemplary embodiment, mixing the BBAC and the DABCO with the alcoholic solvent may include mixing the BBAC and the DABCO with the alcoholic solvent at an equimolar ratio of the BBAC to the DABCO. In an exemplary embodiment, mixing the BBAC and the DABCO with the alcoholic solvent may include mixing the BBAC and the DABCO with the alcoholic solvent at a temperature between room temperature and about 65° C. for a time period between about 0.5 hour and about 72 hours.

In an exemplary embodiment, mixing the BBAC and the DABCO with the alcoholic solvent may include mixing the BBAC with a concentration between about 0.01 mM and about 5 mM and the DABCO with a concentration between about 0.01 mM and about 5 mM with the alcoholic solvent. In an exemplary embodiment, mixing the BBAC and the DABCO with the alcoholic solvent may include stirring the BBAC and the DABCO with the alcoholic solvent using a stirrer. In an exemplary embodiment, mixing the BBAC and the DABCO with the alcoholic solvent may include mixing the BBAC and the DABCO with at least one of methanol, ethanol, and isopropanol.

In an exemplary embodiment, removing the alcoholic solvent from the mixture may include removing the alcoholic solvent from the mixture using a rotary evaporator. In an exemplary embodiment, washing the precipitate with the organic may include washing the precipitate with at least one of ethyl acetate, acetone, chloroform, and diethyl ether. In an exemplary embodiment, synthesizing the multifunctional copolymeric nanoparticle may further include forming a homogenous multifunctional copolymeric nanoparticle by filtering the purified multifunctional copolymeric nanoparticle. In an exemplary embodiment, filtering the purified multifunctional copolymeric nanoparticle may include filtering the purified multifunctional copolymeric nanoparticle using a syringe filter with a pore size of about 220 nm.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A illustrates a schematic representation of an exemplary multifunctional copolymeric nanoparticle, consistent with one or more exemplary embodiments of the present disclosure.

FIG. 1B illustrates a schematic representation of an exemplary method for synthesizing an exemplary multifunctional copolymeric nanoparticle, consistent with one or more exemplary embodiments of the present disclosure.

FIG. 2 illustrates a flowchart of an exemplary method for synthesizing an exemplary multifunctional copolymeric nanoparticle, consistent with one or more exemplary embodiments of the present disclosure.

FIG. 3A illustrates a scanning electron microscope (SEM) image 300 of exemplary multifunctional copolymeric nanoparticles synthesized with a reaction time of about 72 hours, consistent with one or more exemplary embodiments of the present disclosure.

FIG. 3B illustrates an SEM image 302 of exemplary multifunctional copolymeric nanoparticles synthesized with a reaction time of about 24 hours, consistent with one or more exemplary embodiments of the present disclosure.

FIG. 3C illustrates an SEM image 304 of exemplary multifunctional copolymeric nanoparticles synthesized with a reaction time of about 8 hours, consistent with one or more exemplary embodiments of the present disclosure.

FIG. 3D illustrates an SEM image 306 of exemplary multifunctional copolymeric nanoparticles synthesized with a reaction time of about 1 hour, consistent with one or more exemplary embodiments of the present disclosure.

FIG. 4A illustrates a graph 400 for dynamic light scattering (DLS) analysis of exemplary multifunctional copolymeric nanoparticles with a reaction time of about 2 hours, consistent with one or more exemplary embodiments of the present disclosure.

FIG. 4B illustrates a graph 402 for DLS analysis of exemplary multifunctional copolymeric nanoparticles with a reaction time of about 1 hour, consistent with one or more exemplary embodiments of the present disclosure.

FIG. 4C illustrates a graph 404 for DLS analysis of exemplary multifunctional copolymeric nanoparticles with a reaction time of about 30 minutes, consistent with one or more exemplary embodiments of the present disclosure.

FIG. 5A illustrates a permeable gel chromatography (GPC) chromatogram 500 of exemplary multifunctional copolymeric nanoparticles synthesized with a reaction time of about 4 hours, consistent with one or more exemplary embodiments of the present disclosure.

FIG. 5B illustrates a GPC chromatogram 502 of exemplary multifunctional copolymeric nanoparticles synthesized with a reaction time of about 1 hour, consistent with one or more exemplary embodiments of the present disclosure.

FIG. 6A illustrates an image 600 of proton nuclear magnetic resonance (¹H NMR) spectra of exemplary multifunctional copolymeric nanoparticles, consistent with one or more exemplary embodiments of the present disclosure.

FIG. 6B illustrates an image 602 of carbon-13 nuclear magnetic resonance (¹³CNMR) (DMSO d⁶) spectra of exemplary multifunctional copolymeric nanoparticles, consistent with one or more exemplary embodiments of the present disclosure.

FIG. 7 illustrates an image 700 of Fourier-transform infrared spectroscopy (FTIR) spectra of exemplary multifunctional copolymeric nanoparticles, consistent with one or more exemplary embodiments of the present disclosure.

FIG. 8 illustrates a chart 800 for zeta potentials of exemplary multifunctional copolymeric nanoparticles, consistent with one or more exemplary embodiments of the present disclosure.

FIG. 9A illustrates an image 900 of gel retardation assay of different multifunctional copolymeric nanoparticles synthesized with a reaction time of about 19 hours and 72 hours at mass ratios of 1 and 5 (multifunctional copolymeric nanoparticles to the plasmid DNA) in the presence or absence of dithiothreitol (DTT), consistent with one or more exemplary embodiments of the present disclosure.

FIG. 9B illustrates an image 902 of gel retardation assay of different multifunctional copolymeric nanoparticles synthesized with a reaction time of about 30 minutes at a temperature of about 60° C. or room temperature (r.t.) in mass ratios of 1 and 5 (multifunctional copolymeric nanoparticles to the plasmid DNA), consistent with one or more exemplary embodiments of the present disclosure.

FIG. 9C illustrates an image 904 of gel retardation assay of exemplary multifunctional copolymeric nanoparticles synthesized with a reaction time of about 30 minutes at a temperature of about 60° C. or room temperature (r.t.) in minimal mass ratios of 1, 0.3, 0.1, and 0.05 (multifunctional copolymeric nanoparticles to the plasmid DNA), consistent with one or more exemplary embodiments of the present disclosure.

FIG. 10A illustrates a flow cytometry image 1000 for GFP expression of HEK293 cells 48 hours after transfection with multifunctional copolymeric nanoparticles and negative control, consistent with one or more exemplary embodiments of the present disclosure.

FIG. 10B illustrates a chart 1002 for quantification of flow cytometry data of GFP expression after transfection with different ratios of multifunctional copolymeric nanoparticles and plasmid DNA, consistent with one or more exemplary embodiments of the present disclosure.

FIG. 11 illustrates a chart 1100 of antibacterial activity of exemplary multifunctional copolymeric nanoparticles synthesized in different reaction times (1, 3, 8, 24, and 72 hours), consistent with one or more exemplary embodiments of the present disclosure.

FIG. 12 illustrates a chart 1200 for cytotoxicity evaluation of exemplary multifunctional copolymeric nanoparticles 48 hours after exposure to HEK293 cell, consistent with one or more exemplary embodiments of the present disclosure.

FIG. 13A illustrates a graph 1300 for evaluation of antibiofilm activity of exemplary multifunctional copolymeric nanoparticles synthesized with a reaction time of 1 hour (AX 1 h) on Enterococcus, consistent with one or more exemplary embodiments of the present disclosure.

FIG. 13B illustrates a graph 1302 for evaluation of antibiofilm activity of exemplary multifunctional copolymeric nanoparticles synthesized with a reaction time of 1 hour (AX 1 h) on Staphylococcus, consistent with one or more exemplary embodiments of the present disclosure.

FIG. 13C illustrates a graph 1304 for evaluation of antibiofilm activity of exemplary multifunctional copolymeric nanoparticles synthesized with a reaction time of 1 hour (AX 1 h) on Pseudomonas aeruginosa, consistent with one or more exemplary embodiments of the present disclosure.

FIG. 14A illustrates an image 1400 representative of wound healing from days 5 to 21 in local treatment groups, including G2 (P. aeruginosa+AX 1 h) and G3 (S. aureus+AX 1 h) compared with untreated groups as controls, including G1 (P. aeruginosa infection and G4 (S. aureus infection) on mice, consistent with one or more exemplary embodiments of the present disclosure.

FIG. 14B illustrates a plot 1402 for wound area values from days 5 to 21 in local treatment groups, including G2 (P. aeruginosa+AX 1 h) and G3 (S. aureus+AX 1 h) compared with untreated groups as controls, including G1 (P. aeruginosa infection) and G4 (S. aureus infection) on mice, consistent with one or more exemplary embodiments of the present disclosure.

FIG. 15A illustrates a plot 1500 for bacterial load of burn wounds on day 5 in mice intradermally infected with P. aeruginosa and S. aureus (n=4 wounds per group), consistent with one or more exemplary embodiments of the present disclosure.

FIG. 15B illustrates a plot 1502 for bacterial load of burn wounds on day 10 in mice intradermally infected with P. aeruginosa and S. aureus (n=4 wounds per group), consistent with one or more exemplary embodiments of the present disclosure. FIG. 16A illustrates a plot 1600 for plasma levels of interleukin 1 (IL-1) and interleukin 6 (IL-6) after 21 days of wound infection with S. aureus in groups treated with exemplary multifunctional copolymeric nanoparticles synthesized with a reaction time of 1 hour (AX 1 h) and untreated groups, consistent with one or more exemplary embodiments of the present disclosure.

FIG. 16B illustrates a plot 1602 for plasma levels of interleukin 1 (IL-1) and interleukin 6 (IL-6) after 21 days of wound infection with P. aeruginosa in groups treated with exemplary multifunctional copolymeric nanoparticles synthesized with a reaction time of 1 hour (AX 1 h) and untreated groups, consistent with one or more exemplary embodiments of the present disclosure. (n=12-15 per group).

### DETAILED DESCRIPTION

In the following detailed description, numerous specific details are set forth by way of examples in order to provide a thorough understanding of the relevant teachings. However, it should be apparent that the present teachings may be practiced without such details. In other instances, well-known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown but is to be accorded the widest possible scope consistent with the principles and features disclosed herein.

The present disclosure generally describes an exemplary multifunctional copolymeric nanoparticle that may be used as an intelligent carrier with minimal toxicity, positive surface charge, DNA-interacting groups, and small particle size. Exemplary multifunctional copolymeric nanoparticles may highly interact with nucleic acids and may be used as a non-viral carrier for gene delivery and clustered regularly interspaced short palindromic repeats (CRISPR) delivery. Also, exemplary multifunctional copolymeric nanoparticles may be considered a suitable antibacterial agent for eradicating multidrug-resistant bacterial infection.

Exemplary multifunctional copolymeric nanoparticles may include a plurality of 1,4-diazabicyclo[2.2.2]octane (DABCO) monomers crosslinked with a plurality of bromoacetyl cystamine (BBAC) monomers. In the present disclosure, crosslinking may be defined as the process of forming covalent bonds or relatively short sequences of chemical bonds to join two DABCO monomers together. In an exemplary embodiment, each of the plurality of DABCO monomers and each of the plurality of BBAC monomers may alternate in sequence. In an exemplary embodiment, each of the plurality of DABCO monomers and each of the plurality of BBAC monomers may be repeated after each other in a sequence.

In an exemplary embodiment, the multifunctional copolymeric nanoparticle may have a formula:

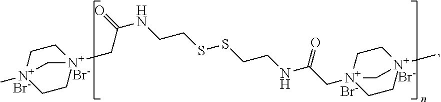

wherein n is an integer between 5 and 40. In an exemplary embodiment, the multifunctional copolymeric nanoparticle may have a particle size between about 1 nm and about 2 μm.

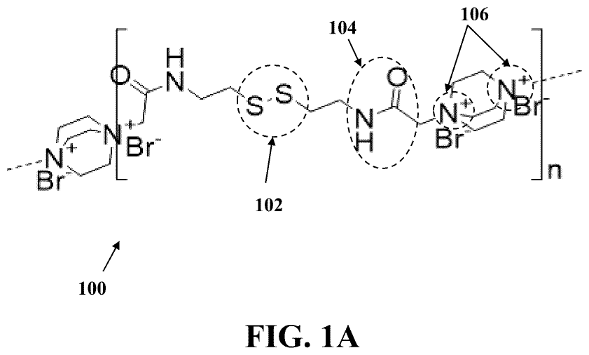

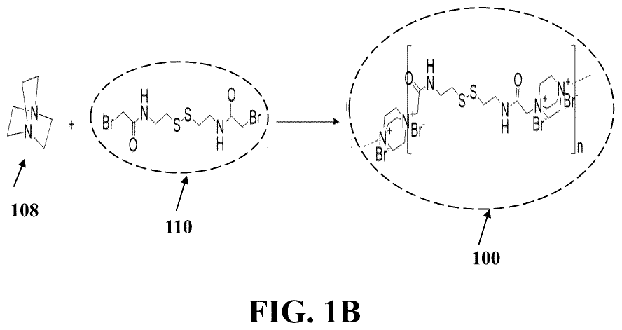

In the present disclosure, exemplary multifunctional copolymeric nanoparticles may refer to copolymeric nanoparticles with different functional groups. FIG. 1A illustrates a schematic representation of exemplary multifunctional copolymeric nanoparticle 100, consistent with one or more exemplary embodiments of the present disclosure. Referring to FIG. 1A, exemplary multifunctional copolymeric nanoparticle 100 may have different functional groups, including a disulfide group 102, an amide group 104, and an ammonium group 106.

In an exemplary embodiment, the multifunctional copolymeric nanoparticle 100 may be a redox-sensitive copolymeric nanoparticle due to the presence of disulfide group 102 which may be cleaved into two thiol groups on an reducing environment. As a result of the presence of disulfide group 102 in the structure of an exemplary multifunctional copolymeric nanoparticle 100, the multifunctional copolymeric nanoparticle may release nucleic acid or drug inside a reducing environment, such as an exemplary cells' reducing environment, which has a high concentration of glutathione (GSH). In an exemplary embodiment, cleavage of disulfide group 102 into two thiol groups in an exemplary reducing environment of exemplary cells may lead to cargo release from exemplary multifunctional copolymeric nanoparticle 100 as a non-viral vector inside exemplary cells.

In an exemplary embodiment, multifunctional copolymeric nanoparticle 100 may be a cationic multifunctional copolymeric nanoparticle. In an exemplary embodiment, multifunctional copolymeric nanoparticle 100 may be a transfection reagent for delivering a nucleic acid molecule into cells. In an exemplary embodiment, the interaction of exemplary multifunctional copolymeric nanoparticle 100 with nucleic acids may be based on a negative charge of exemplary nucleic acids and a positive charge of exemplary multifunctional copolymeric nanoparticle and formation of multiple electrostatic bonds. In an exemplary embodiment, exemplary multifunctional copolymeric nanoparticle 100 may have a positive charge and different functional groups that may interact with nucleic acids.

In an exemplary embodiment, an exemplary nucleic acid molecule may include at least one of deoxyribonucleic acid (DNA) and ribonucleic acid (RNA). In an exemplary embodiment, an exemplary nucleic acid molecule may include at least one of a plasmid, siRNA, miRNA, and mRNA. In an exemplary embodiment, a ratio of an exemplary nucleic acid molecule to an exemplary multifunctional copolymeric nanoparticle may be between about 1:1 and about 1:200.

In an exemplary embodiment, multifunctional copolymeric nanoparticle 100 may have antibacterial and anti-biofilm activity. Although the mammalian cell membrane is zwitterionic, the bacterial cell membrane has a negative charge. As a result, exemplary multifunctional copolymeric nanoparticles with positive charge may target an exemplary bacterial cell membrane, which has an immense role in the survival of bacteria and may be considered a suitable antibacterial agent with excellent antibacterial activity and low toxicity on mammalian cells. In an exemplary embodiment, multifunctional copolymeric nanoparticle 100 may have antibacterial activity against gram-positive and gram-negative bacteria. In an exemplary embodiment, gram-positive bacteria may include Staphylococcus aureus, Enterococcus. In an exemplary embodiment, gram-negative bacteria may include Pseudomonas aeruginosa and Escherichia coli.

Exemplary multifunctional copolymeric nanoparticle 100 may be synthesized using a simple and highly efficient method without requiring sophisticated techniques, initiators, or reducing agents. Exemplary multifunctional copolymeric nanoparticle 100 may be synthesized through a free-radical addition polymerization (FRAP) method by reacting 1,4-diazabicyclo[2.2.2]octane (DABCO) with bromoacetyl cystamine (BBAC) as a crosslinker. In an exemplary embodiment, exemplary method 200 may have several advantages, including ease of operation, low number of synthesis steps, and lack of difficult separation and purification steps such as chromatography.

In an exemplary embodiment, utilizing exemplary BBAC as a crosslinker offers several benefits. For instance, there is no need to use precious metal catalysts and expensive primers such as azobisisobutyronitrile (AIBN), toxic additives, and neutral environments like argon gas or nitrogen. Also, BBAC crosslinker may allow an incremental polymerization process to be done in an elementary and hassle-free condition. While dark conditions or neutral gases are usually required for polymerizable reactions that are either radical and are initiated or proceeded through metal catalysts, exemplary multifunctional copolymeric nanoparticle 100 is not sensitive to light. Also, exemplary multifunctional copolymeric nanoparticle 100 may strongly interact with nucleic acids even after one month when kept at laboratory temperature or after six months when refrigerated. Also, exemplary method 200 may be easily carried out in the air.

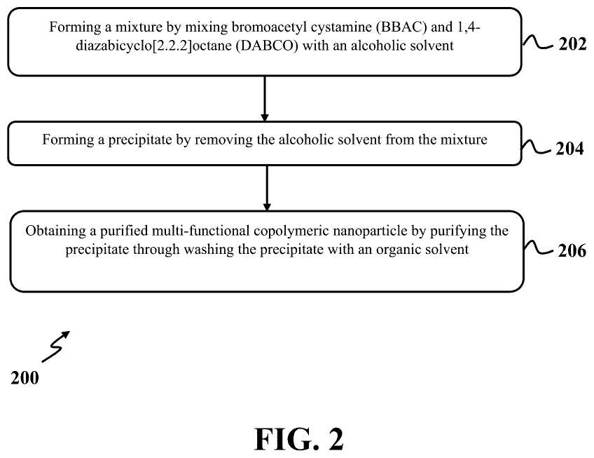

FIG. 2 illustrates a flowchart of an exemplary method 200 for synthesizing an exemplary multifunctional copolymeric nanoparticle, consistent with one or more exemplary embodiments of the present disclosure. Exemplary method 200 may include forming a mixture by mixing bromoacetyl cystamine (BBAC) and 1,4-diazabicyclo[2.2.2]octane (DABCO) with an alcoholic solvent (step 202), forming a precipitate by removing the alcoholic solvent from the mixture (step 204) and obtaining a purified multifunctional copolymeric nanoparticle by purifying the precipitate through washing the precipitate with an organic solvent (step 206).

In further detail with respect to step 202, in an exemplary embodiment, forming a mixture by mixing BBAC and DABCO with an alcoholic solvent may include mixing the BBAC and the DABCO with the alcoholic solvent at an equimolar ratio of the BBAC to the DABCO. FIG. 1B illustrates a schematic representation of step 202 of exemplary method 200 for synthesizing exemplary multifunctional copolymeric nanoparticle 100 by mixing DABCO 108 and BBAC 110 with an alcoholic solvent, consistent with one or more exemplary embodiments of the present disclosure.

In an exemplary embodiment, mixing DABCO 108 and BBAC 110 with the alcoholic solvent may include mixing DABCO 108 and BBAC 110 with the alcoholic solvent at a temperature between room temperature and about 65° C. In an exemplary embodiment, mixing DABCO 108 and BBAC 110 with the alcoholic solvent may include mixing DABCO 108 and BBAC 110 with the alcoholic solvent for a reaction time between about 0.5 hour and about 72 hours. In an exemplary embodiment, lower reaction times may lead to smaller multifunctional copolymeric nanoparticles. In an exemplary embodiment, a reaction time of about 1 hour may lead to exemplary multifunctional copolymeric nanoparticles with a particle size of about 25 nm. In an exemplary embodiment, mixing DABCO 108 and BBAC 110 with the alcoholic solvent may include mixing DABCO 108 and BBAC 110 with at least one of methanol, ethanol, and isopropanol.

In an exemplary embodiment, mixing DABCO 108 and BBAC 110 with the alcoholic solvent may include mixing BBAC 110 with a concentration between about 0.01 mM and about 5 mM and DABCO 108 with a concentration between about 0.01 mM and about 5 mM with the alcoholic solvent. In an exemplary embodiment, mixing DABCO 108 and BBAC 110 with the alcoholic solvent may include stirring DABCO 108 and BBAC 110 with the alcoholic solvent using a stirrer.

In further detail with respect to step 204, in an exemplary embodiment, forming a precipitate may include removing the alcoholic solvent from the mixture. In an exemplary embodiment, removing the alcoholic solvent from the mixture may include removing the alcoholic solvent from the mixture using a rotary evaporator. In an exemplary embodiment, removing the alcoholic solvent from the mixture using the rotary evaporator may include removing the alcoholic solvent from the mixture through evaporation of the alcoholic solvent under reduced pressure.

In further detail with respect to step 206, in an exemplary embodiment, obtaining a purified multifunctional copolymeric nanoparticle may include purifying the precipitate by washing the precipitate with an organic solvent. In an exemplary embodiment, washing the precipitate with the organic solvent may include removing impurities which are soluble in the organic solvent. In an exemplary embodiment, washing the precipitate with the organic may include washing the precipitate with at least one of ethyl acetate, acetone, chloroform, and diethyl ether. In an exemplary embodiment, obtaining the purified multifunctional copolymeric nanoparticle may include obtaining the purified multifunctional copolymeric nanoparticle with about 99% efficiency, making exemplary method 200 capable of large-scale production.

Since multifunctional copolymeric nanoparticle 100 is highly aqueous in contrast to DABCO 108 and BBAC 110, which are organic substances, multifunctional copolymeric nanoparticle 100 as the desired product may be readily purified by mixing with an organic solvent, such as ethyl acetate and chloroform without any need for dialysis bag during purification. As a result, there is no need for expensive and time-consuming methods and equipment to purify multifunctional copolymeric nanoparticle 100.

In an exemplary embodiment, an exemplary method for synthesizing the multifunctional copolymeric nanoparticle may further include forming a homogenous multifunctional copolymeric nanoparticle by filtering the purified multifunctional copolymeric nanoparticle using a syringe filter with a pore size of about 220 nm. In an exemplary embodiment, an exemplary homogenous multifunctional copolymeric nanoparticle may be used for gene delivery purposes.

### EXAMPLES

### Example 1: Physical Characterization of Exemplary Multifunctional Copolymeric Nanoparticles

In this example, exemplary multifunctional copolymeric nanoparticles were synthesized utilizing a process similar to exemplary method 200 as presented in FIG. 2, and their physical properties were characterized. At first, about 1 mmol of DABCO and about 1 mmol of BBAC were poured into a test tube containing methanol with a volume from about 2 ml to about 10 ml. After that, the test tube was placed on a steerer at a temperature of about 60° C. for different time periods between 30 minutes to 72 hours. Finally, methanol was evaporated using a rotary apparatus, and the precipitate was washed twice with about 2 ml of ethyl acetate to obtain the desired multifunctional copolymeric nanoparticles with 99% efficiency.

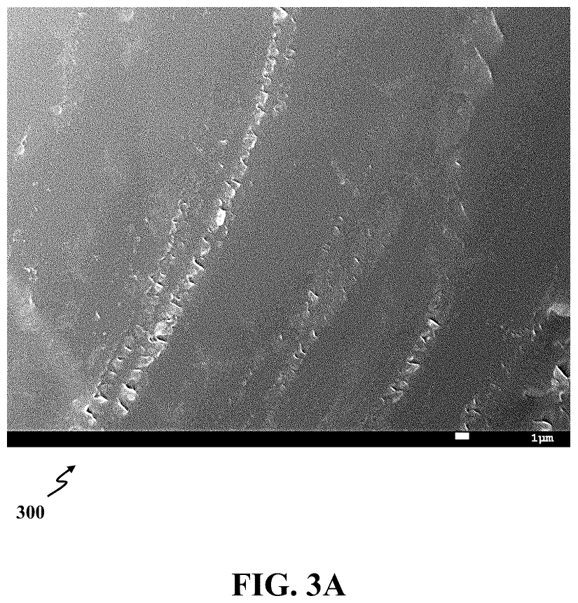

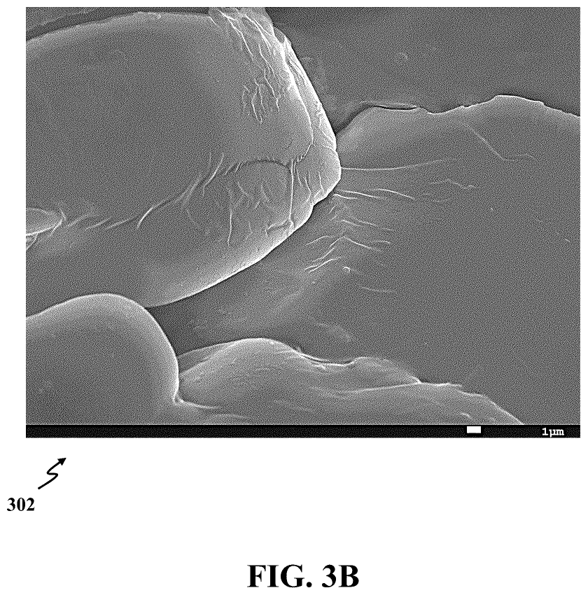

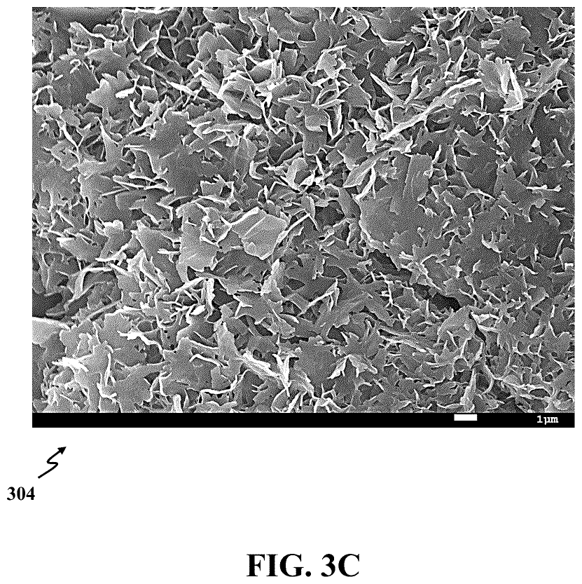

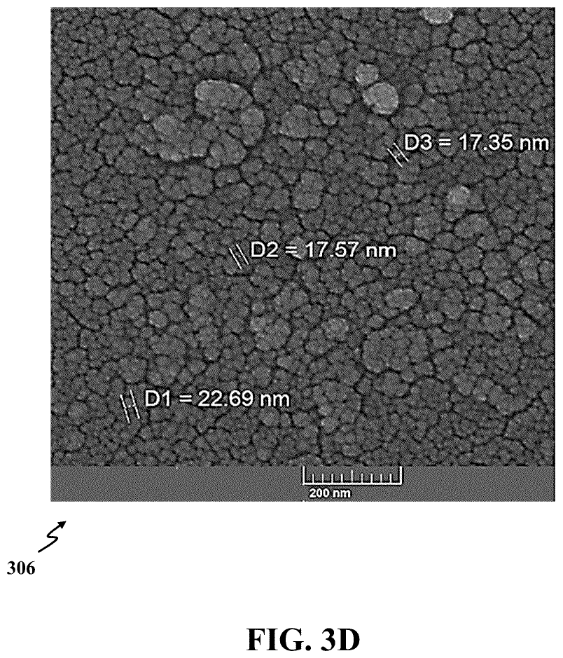

After synthesis, different analyses were done to characterize the multifunctional copolymeric nanoparticles, including Scanning electron microscopy (SEM) analysis for determining surface morphology, dynamic light scattering (DLS) for determining particle size, and permeable gel chromatography (GPC) for determining molecular mass. FIG. 3A illustrates a scanning electron microscope (SEM) image 300 of exemplary multifunctional copolymeric nanoparticles synthesized with a reaction time of about 72 hours, consistent with one or more exemplary embodiments of the present disclosure. FIG. 3B illustrates an SEM image 302 of exemplary multifunctional copolymeric nanoparticles synthesized with a reaction time of about 24 hours, consistent with one or more exemplary embodiments of the present disclosure.

FIG. 3C illustrates an SEM image 304 of exemplary multifunctional copolymeric nanoparticles synthesized with a reaction time of about 8 hours, consistent with one or more exemplary embodiments of the present disclosure. FIG. 3D illustrates an SEM image 306 of exemplary multifunctional copolymeric nanoparticles synthesized with a reaction time of about 1 hour, consistent with one or more exemplary embodiments of the present disclosure. Referring to FIGS. 3A-3D, SEM images 300-306 of the exemplary multifunctional copolymeric nanoparticles indicate that the size of the exemplary multifunctional copolymeric nanoparticles decreases sharply with decreasing reaction time.

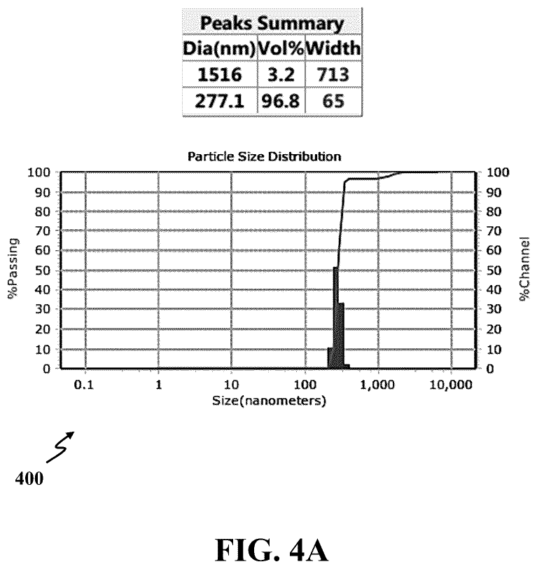

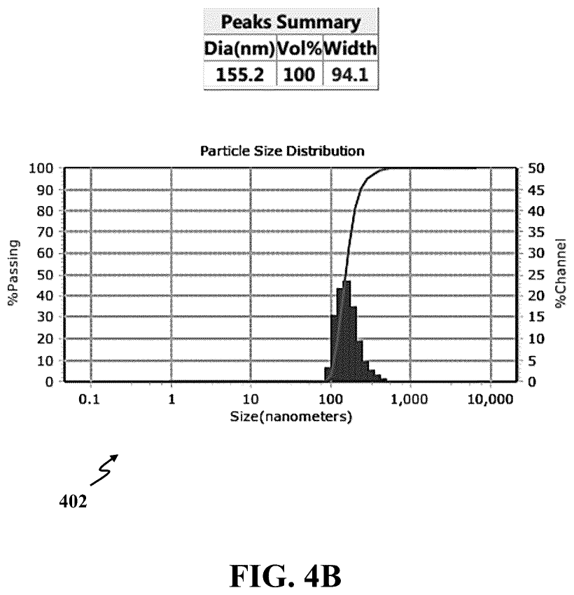

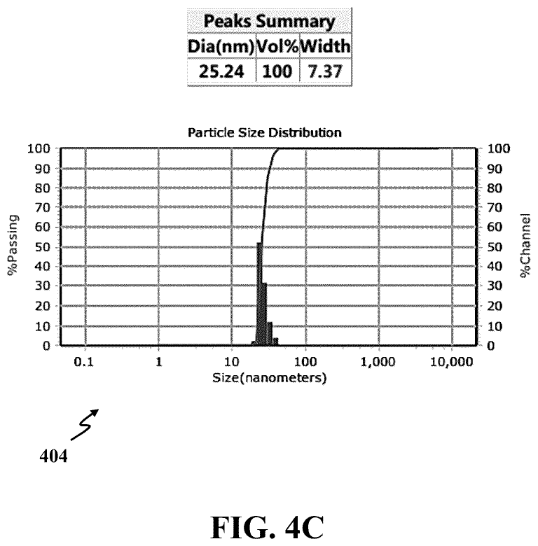

Also, a DLS test was used to evaluate the particle size of exemplary multifunctional copolymeric nanoparticles with their size distribution. FIG. 4A illustrates a graph 400 of DLS analysis of exemplary multifunctional copolymeric nanoparticles with a reaction time of about 2 hours, consistent with one or more exemplary embodiments of the present disclosure. FIG. 4B illustrates a graph 402 of DLS analysis of exemplary multifunctional copolymeric nanoparticles with a reaction time of about 1 hour, consistent with one or more exemplary embodiments of the present disclosure.

FIG. 4C illustrates a graph 404 or DLS analysis of exemplary multifunctional copolymeric nanoparticles with a reaction time of about 30 minutes, consistent with one or more exemplary embodiments of the present disclosure. Referring to FIGS. 4A-4C, graphs 400-404 illustrating DLS results confirm that the size of the exemplary multifunctional copolymeric nanoparticles decreases sharply with decreasing reaction time. Also, the long, narrow DLS curves confirm the ability of exemplary methods of the present disclosure to produce uniform nanoparticles with the same size of 277 nm, 150 nm, and 25 nm in reaction times of 2 hours, 1 hour, and 30 minutes, respectively.

Moreover, GPC analysis was used to investigate the molecular mass of exemplary multifunctional copolymeric nanoparticles synthesized at different reaction times. Permeable gel chromatography (GPC) is a desirable analytical tool for identifying natural and synthetic polymers and proteins that use porous gel particles to separate polymers in solution. An exemplary solution containing an exemplary multifunctional copolymeric nanoparticle is pumped into a column containing porous particles. As a solution containing an exemplary multifunctional copolymeric nanoparticles passes through the column, small molecules penetrate the pores of the column filler and, as a result, exit with a time delay relative to the larger molecules. The molecular weight of the test samples is relative and is obtained by comparing its exit time with the calibration curve.

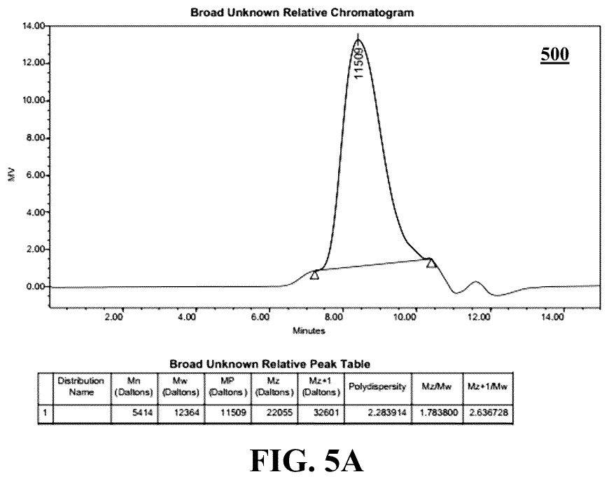

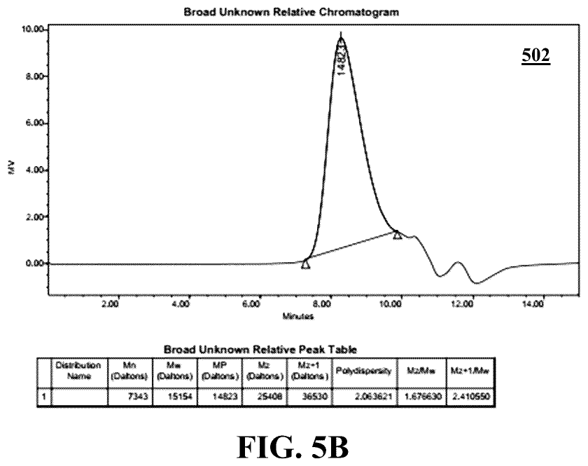

FIG. 5A illustrates a permeable gel chromatography (GPC) chromatogram 500 of exemplary multifunctional copolymeric nanoparticles synthesized with a reaction time of about 4 hours, consistent with one or more exemplary embodiments of the present disclosure. FIG. 5B illustrates a GPC chromatogram 502 of exemplary multifunctional copolymeric nanoparticles synthesized with a reaction time of about 1 hour, consistent with one or more exemplary embodiments of the present disclosure. Referring to FIGS. 5A-5B, chromatograms 500-502 illustrate that reaction time of about 1 hour lead to exemplary multifunctional copolymeric nanoparticles with a molecular weight of about 12 KDa. However, exemplary multifunctional copolymeric nanoparticles synthesized with a reaction time of about 4 hours have a molecular weight of about 15 KDa. As a result, the molecular weight of the exemplary multifunctional copolymeric nanoparticles decreases with decreasing reaction time.

### Example 2: Chemical Characteristics of Exemplary Multifunctional Copolymeric Nanoparticles

In this example, chemical characterization of exemplary multifunctional copolymeric nanoparticles was done using nuclear magnetic resonance (NMR) spectroscopy to identify organic compounds' structure, Fourier-transform infrared spectroscopy (FTIR) to determine the presence of functional functionalism groups, and zeta potential analysis for determining the surface charge.

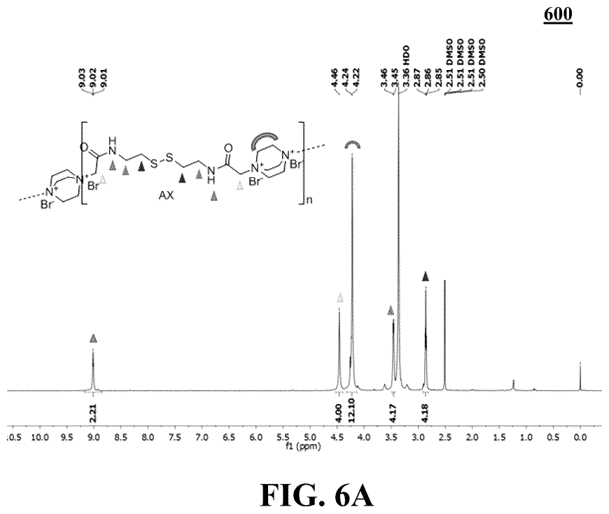

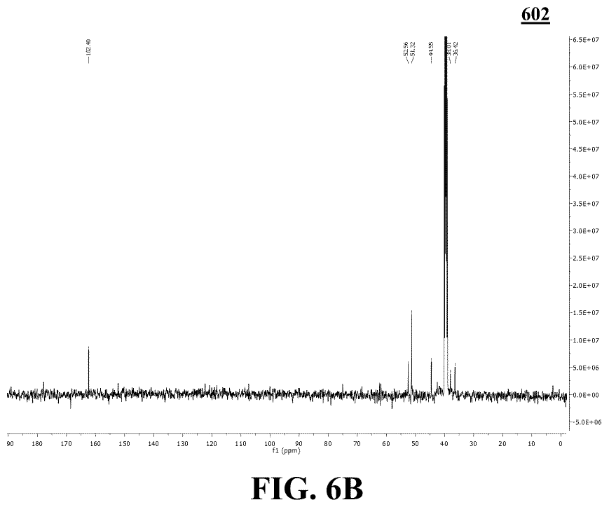

As for chemical and biological analyses, NMR analysis was used to ensure the synthesis of exemplary multifunctional copolymeric nanoparticles. FIG. 6A illustrates an image 600 of the proton nuclear magnetic resonance (¹H NMR) spectra of exemplary multifunctional copolymeric nanoparticles, consistent with one or more exemplary embodiments of the present disclosure. Referring to FIG. 6A, the ¹H NMR spectra of image 600 proves the synthesis of the exemplary multifunctional copolymeric nanoparticles as the desired product. There are 12 hydrogens related to DABCO heterocycles in the range of 4.22 to 4.24, and hydrogens related to amides in the range of 9.01 to 9.03. There are three methylene hydrogen regions with sub-peak levels of 4 hydrogens in areas 2.86, 3.46, and 4.46. Examination of the ¹H NMR spectrum fully confirms that exemplary copolymeric nanoparticles have been successfully synthesized.

FIG. 6B illustrates an image 602 of representative carbon-13 nuclear magnetic resonance (¹³CNMR) (DMSO d⁶) spectra of exemplary multifunctional copolymeric nanoparticles, consistent with one or more exemplary embodiments of the present disclosure. Referring to FIG. 6B, the ¹³C NMR spectra of image 602 of exemplary multifunctional copolymeric nanoparticles indicates the most prominent peaks, including the amide peak in 162 and four peaks related to methylene carbons.

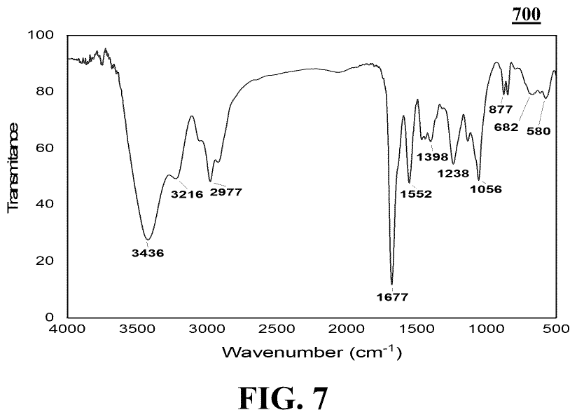

FIG. 7 illustrates an image 700 Fourier-transform infrared spectroscopy (FTIR) spectrum of exemplary multifunctional copolymeric nanoparticles, consistent with one or more exemplary embodiments of the present disclosure. Referring to FIG. 7, the peak in region 877 cm⁻¹ belongs to the disulfide functional group of exemplary multifunctional copolymeric nanoparticles, and the peak in region 580 cm⁻¹ is for the carbon-sulfur bond. Further, the presence of a sharp peak in the area of 1677 cm⁻¹ confirms the existence of amide groups in exemplary multifunctional copolymeric nanoparticles. There is also a carbon-hydrogen bond with a sharp, long peak in the 2977 cm⁻¹ region. In addition, the broad peak in region 3436 cm⁻¹ confirms the presence of amine groups in exemplary multifunctional copolymeric nanoparticles.

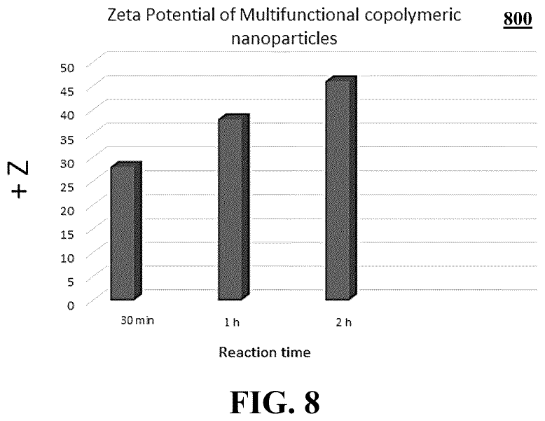

Moreover, Zeta analysis was also used to determine the surface charge and the positive charge of the exemplary multifunctional copolymeric nanoparticles. FIG. 8 illustrates a chart 800 of zeta potentials of exemplary multifunctional copolymeric nanoparticles, consistent with one or more exemplary embodiments of the present disclosure. Referring to FIG. 8, results of zeta analysis indicate that the surface charge of exemplary multifunctional copolymeric nanoparticles is highly positive; as a result, exemplary multifunctional copolymeric nanoparticles are cationic.

### Example 3: DNA Interaction of Exemplary Multifunctional Copolymeric Nanoparticles

Electrophoresis and gel retardation assay is a laboratory method for analyzing and separating macromolecules based on their size and load. In electrophoresis and gel retardation assay, by trapping DNA in exemplary multifunctional copolymeric nanoparticles, the ability of DNA to move on gel electrophoresis may be lost. The complex formation between DNA and the exemplary multifunctional copolymeric nanoparticles was examined using different mass ratios of the exemplary multifunctional copolymeric nanoparticles as the carrier to the genetic content (W:W).

Obviously, as the size of the nanoparticle becomes smaller, or in other words, in the lower mass ratio of the carrier to the genetic content, more DNA maintenance may be a sign of superiority of the structure and stronger interaction of the structure with the genetic material. For this assay, different mass ratios (0.05, 0.1, 0.3, 1, and 5) were made by combining the exemplary multifunctional copolymeric nanoparticles with certain nanograms of plasmid DNA, and it took 30 minutes for the complex of multifunctional copolymeric nanoparticles and the plasmid DNA to be formed.

After loading multifunctional copolymeric nanoparticles containing the plasmid into the lanes of agarose gel, the samples were electrophoresed at 80 volts, and after 30 minutes, free DNA or DNA that interacts weakly with the carrier began to release. There is a goal to build a nanocarrier that can interact strongly with DNA and prevent DNA from being released outside the cell. Finally, the bands resulting from DNA release were exposed using ultraviolet light in the gel dock device (gel image recognition system), and the gel was imaged. As a result, the appropriate mass ratio of DNA to nanoparticles was obtained using the electrophoresis gel.

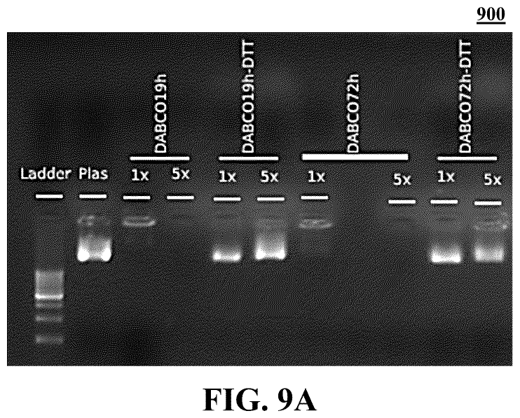

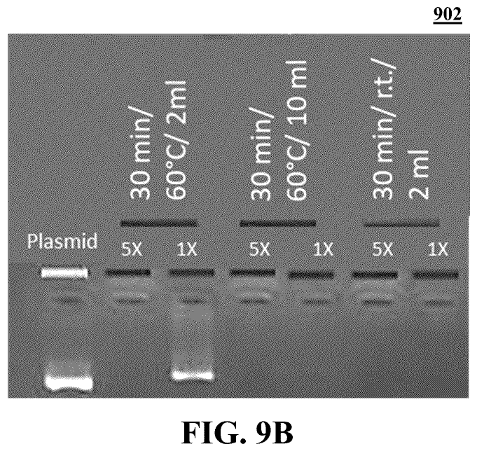

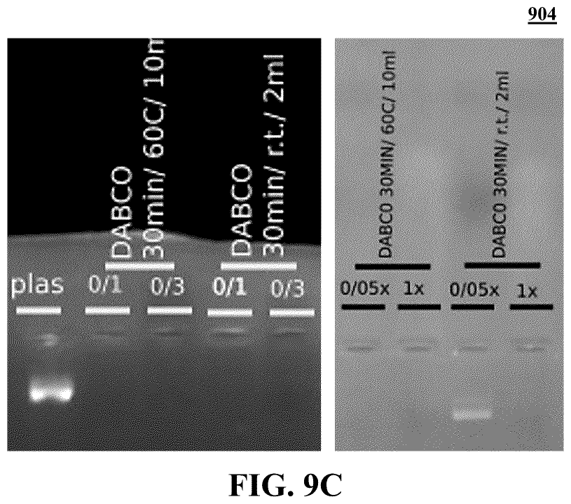

Also, to ensure that multifunctional copolymeric nanoparticles are considered a stimuli-responsive carrier, dithiothreitol (DTT) with the formula C4H10O2S2 was used to reduce the compound for breaking the disulfide bond and thus releasing the plasmid DNA. FIG. 9A illustrates an image 900 of results of gel retardation assay of different multifunctional copolymeric nanoparticles synthesized with a reaction time of about 19 hours and 72 hours at mass ratios of 1 and 5 (multifunctional copolymeric nanoparticles to the plasmid DNA) in the presence or absence of dithiothreitol (DTT), consistent with one or more exemplary embodiments of the present disclosure. Referring to FIG. 9A, both of the multifunctional copolymeric nanoparticles synthesized with a reaction time of about 19 hours and 72 hours at both mass ratios of 1 and 5 can store DNA without any release, and after adding DTT, the results were acceptable, and the intelligent release of DNA occurred.

FIG. 9B illustrates an image 902 of results of gel retardation assay of different multifunctional copolymeric nanoparticles synthesized with a reaction time of about 30 minutes at a temperature of about 60° C. or room temperature (r.t.) and in the presence of 2 ml or 10 ml methanol and used with mass ratios of 1 and 5 (multifunctional copolymeric nanoparticles to the plasmid DNA), consistent with one or more exemplary embodiments of the present disclosure. FIG. 9C illustrates an image 904 of results of gel retardation assay of exemplary multifunctional copolymeric nanoparticles synthesized with a reaction time of about 30 minutes at a temperature of about 60° C. or room temperature (r.t.) in minimal mass ratios of 1, 0.3, 0.1, and 0.05 (multifunctional copolymeric nanoparticles to the plasmid DNA), consistent with one or more exemplary embodiments of the present disclosure.

Referring to FIGS. 9B-9C, images 900-904 illustrate that exemplary multifunctional copolymeric nanoparticles synthesized at 60° C. in 30 minutes and in the presence of 10 ml methanol could effectively store DNA in all examined mass ratios. It is noteworthy that the carrier could still store DNA by making the exemplary multifunctional copolymeric nanoparticles smaller and making carriers with a minimum mass ratio of 0.05. It is sporadic for a carrier to hold DNA up to this mass ratio. The ratio of 0.05 is 40 times lower than that used by conventional commercial and widely used carriers such as polyethyleneimine. As a result, the low mass ratio of 0.05 indicates that exemplary multifunctional copolymeric nanoparticles can interact strongly with DNA (about 40 times more than polyethyleneimine) and prevent DNA from being released outside the cell.

### Example 4: Plasmid Transfection Using Exemplary Multifunctional Copolymeric Nanoparticles

In this example, the transfection efficiency of exemplary multifunctional copolymeric nanoparticles was examined by transfecting a plasmid encoding green fluorescent protein (GFP) to HEK293 cell line using exemplary multifunctional copolymeric nanoparticles and study GFP expression. HEK293 cell line was cultured in DEMEM (high glucose) medium with 10% serum and 1% penicillin-streptomycin in cell culture flask and incubated with humid air and CO₂ concentration (5%). After trypsinizing the cultured cells in the flask, the cells were counted using a hemocytometer slide, and 50,000 cells were transferred to each well of a 24-cell plate culture cell containing 500 μl of 10% serum medium. After a while, when the cell density reached 70% of the well's surface, the cells were ready for transfection.

In the next step, polyplex was prepared by mixing DNA plasmid and multifunctional copolymeric nanoparticles and added to the cells in more than 5 different ratios in the final volume of 50 μl to 150 μl of culture medium. After 4 hours, the cell medium was removed, and a fresh medium containing serum and antibiotics was added to the cells. After 48 hours, the plasmid transfection efficiency was examined qualitatively, and flow cytometry was used to quantitatively evaluate transfection rate and GFP gene expression. Also, polyethyleneimine with a molecular weight of 25 kDa (PEI25) was used as a standard and commercial carrier to transfect cells as the control group.

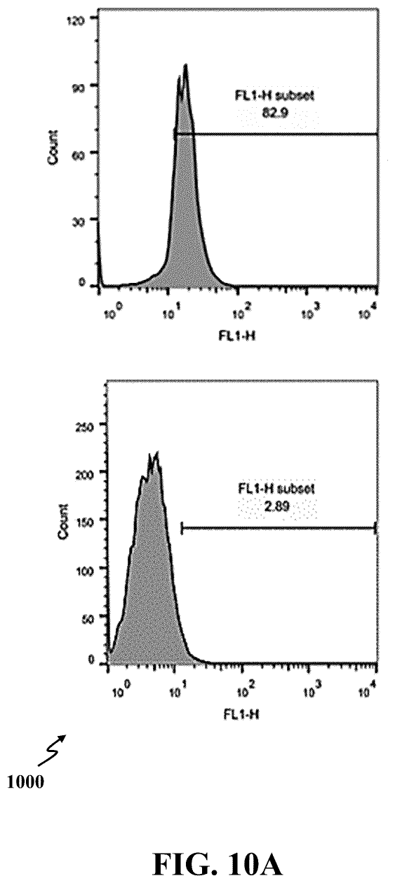

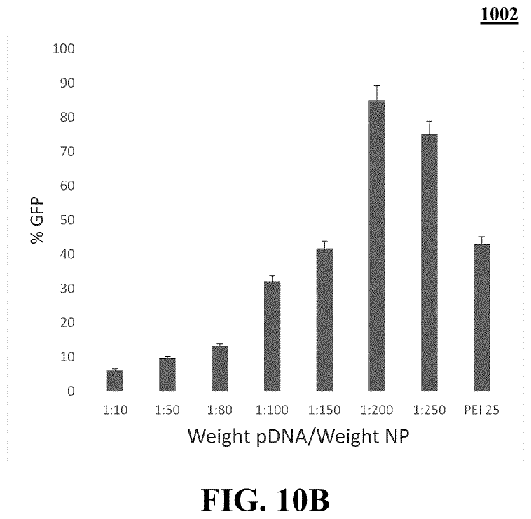

FIG. 10A illustrates an image 1000 for GFP expression of HEK293 cells 48 hours after transfection with multifunctional copolymeric nanoparticles and negative control, consistent with one or more exemplary embodiments of the present disclosure. FIG. 10B illustrates a chart 1002 for quantification of flow cytometry data and evaluation of GFP expression after transfection with different ratios of multifunctional copolymeric nanoparticles and plasmid DNA, consistent with one or more exemplary embodiments of the present disclosure. Referring to FIGS. 10A-10B, the highest transfection rate, which is more than 80%, is observed after 48 hours in HEK293 cells at a weight ratio of 1:200 of multifunctional copolymeric nanoparticles to the plasmid.

It should be noted that high molecular weight polyethylene imines, such as 25 kDa polyethyleneimine, which was used as a comparison in this study, despite its relative efficiency in the gene delivery process, is also highly toxic. Lower molecular weight such as 2 kDa polyethylene imine, despite its low toxicity, cannot transfect the gene properly. However, exemplary multifunctional cationic nanopolymers with disulfide, amide, and cationic groups show all three characteristics of DNA interaction, gene transfection, and low toxicity. Disulfide groups within the cell are ruptured under the influence of reducing agents in target cells, and the rupture causes the carried plasmid to be released inside the cell. In addition, it was found that lacking the presence of a smart, functional group causes the gene expression to be decreased and the carrier toxicity to be increased.

### Example 5: Antibacterial Activity and Cytotoxicity Effect of Exemplary Multifunctional Copolymeric Nanoparticles

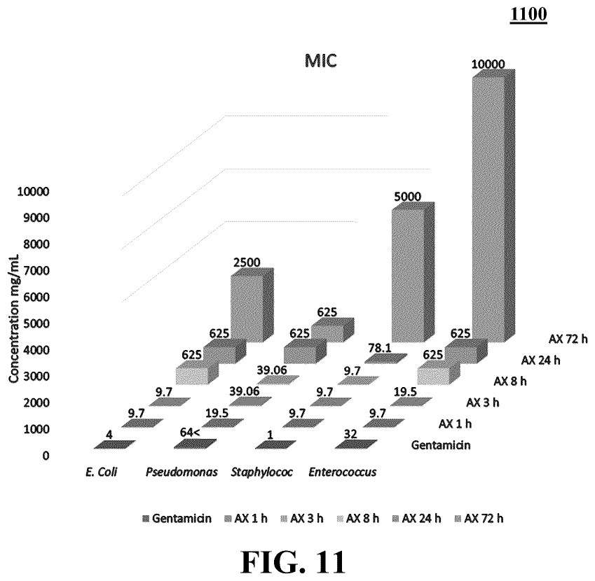

In this example, the antibacterial activity of exemplary multifunctional copolymeric nanoparticles against Gram-negative and Gram-positive strains, including P. aeruginosa, E. coli, S. aureus, and Enterococcus, were investigated based on their minimum bactericidal concentration (MBC) and minimum inhibitory concentration (MIC). Reaction time for synthesis of exemplary multifunctional copolymeric nanoparticles (AX) was varied from 1 hour to 72 hours to fine-tune the antibacterial activity of the exemplary multifunctional copolymeric nanoparticles. FIG. 11 illustrates a chart 1100 for the antibacterial activity of exemplary multifunctional copolymeric nanoparticles synthesized in different reaction times (1, 3, 8, 24, and 72 hours), consistent with one or more exemplary embodiments of the present disclosure.

Referring to FIG. 11, chart 1100 illustrates that a reduction in polymerization time from 72 to 24, 8, 3, and 1 hour affected the MIC results. Interestingly, limiting the time spent for synthesis from 72 hours (AX 72 h) to 24 hours (AX 24 h) caused a sharp drop in MIC toward all the strains, specifically, decreasing the effective concentration from 10000 to 625 μg/mL against Enterococcus and from 5000 to 78.1 μg/mL against S. aureus. As a result of continuing the synthesis time reduction from 8 hours (AX 8 h) to 3 hours (AX 3 h), a significant increase in the antibacterial effect against the Enterococcus (MIC of 625 to 19.5 μg/mL) and E. coli (MIC of 625 to 9.7 μg/mL) is found again.

The best-synthesized exemplary multifunctional copolymeric nanoparticles synthesized with a reaction time of 1 hour (AX 1 h) showed 3.36 times (against pseudomonas) and 3.29 times (against Enterococcus) higher antibacterial activity than gentamycin. Consequently, as the reaction time of synthesis decreased, the antibacterial activity of AX 1 h dramatically increased, which resulted in a higher antibacterial effect against S. aureus, Enterococcus, P. aeruginosa, and E. coli with MIC of 9.7, 9.7, 19.5, and 9.7 μg/mL respectively, in comparison with the MIC of gentamycin, as a standard antibiotic.

It should be noted that P. aeruginosa is a difficult-to-treat gram-negative bacterium that causes many nosocomial infections (hospital-acquired infections) and is known to show resistance to almost all clinically approved drugs. It was also found that although gentamicin failed to have a specific inhibitory effect on P. aeruginosa, even at higher concentrations (>64 μg/mL), the AX 1 h could inhibit the growth of the bacterium with the lowest concentration.

Indeed, an antibacterial agent must be toxic for bacteria but not for human cells. In this respect, AX 1 h impact on the cell viability has been estimated. In this regard, the cytotoxicity effect of AX 1 h was examined by determining the viability of HEK293 cells using the MTT assay under treatment of AX 1 h at lower (0.5 μg/mL) and higher (80 μg/mL) concentrations of effective antibacterial concentration. Normalization of the data was performed based on considering the viability of untreated cells as 100%.

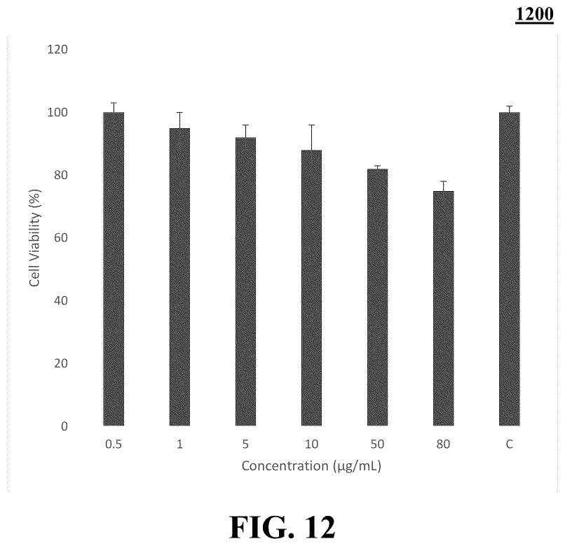

FIG. 12 illustrates a chart 1200 for the cytotoxicity evaluation of exemplary multifunctional copolymeric nanoparticles 48 hours after exposure to HEK293 cell, consistent with one or more exemplary embodiments of the present disclosure. Referring to FIG. 12, chart 1200 illustrates that there is no apparent cytotoxicity at higher concentrations (50 μg/mL and 80 μg/mL) of AX 1 h compared to the control, and the cell viability was about 80% which can be related to the size of the nanoparticle. A decrease in the size of multifunctional copolymeric nanoparticles contributes to a sharp fall in cytotoxicity. The MTT test also showed that these exemplary multifunctional copolymeric nanoparticles had high cell survival and did not show specific toxicity to human cells. However, polyethyleneimine is highly toxic in the concentrations used as carriers.

The results demonstrate that the exemplary multifunctional copolymeric nanoparticles synthesized in shorter times have less toxicity and better bio-applications in gene delivery and antibacterial activity. In general, nanocarriers should have about 200 nm or less particle size. Biological studies showed that the highest bioactivity and the lowest toxicity could be reached with a reaction time of 1 hour, leading to multifunctional copolymeric nanoparticles with a particle size of about 25 nanometers. The results showed that exemplary multifunctional copolymeric nanoparticles with smaller size and lower polydispersity index (PDI), which have better biological properties, can be synthesized by reducing the reaction time and increasing the amount of solvent.

### Example 6: Antibiofilm Activity of Exemplary Multifunctional Polymeric Nanoparticles

Biofilms are aggregated microorganism communities attached to all kinds of surfaces, such as natural materials above and below ground, metals, plastics, medical implant materials, even plant and body tissue and incorporated in a self-produced matrix. In addition, bacteria demonstrate slow metabolism in this protective condition and show 10 to 1000-fold enhanced insensitivity to traditional antibiotic therapy. According to the National Institutes of Health (NIH), bacterial biofilms account for over 80% of humans' infections. The formation of biofilms from gram-positive and negative species is responsible for many serious infections resistant to the human immune system and antibiotic treatments. For instance, S. aureus biofilm causes wound infections, musculoskeletal infections, contact lenses infections, and dental caries. Also, Pseudomonas biofilm is responsible for some respiratory diseases, including ventilator-associated pneumonia (VAP) and cystic fibrosis (CF) lung infections, and about 20% of urinary tract infections occur due to Enterococcus biofilms.

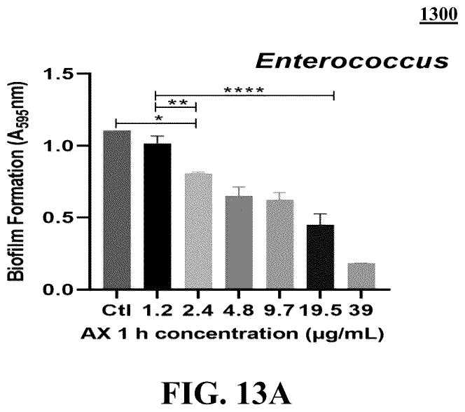

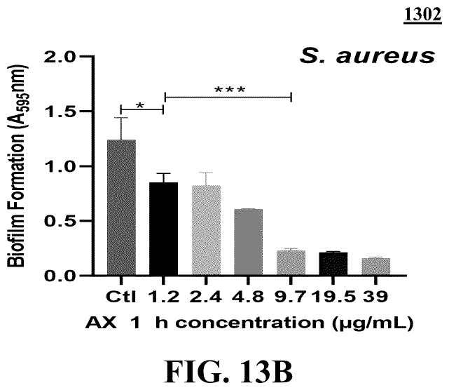

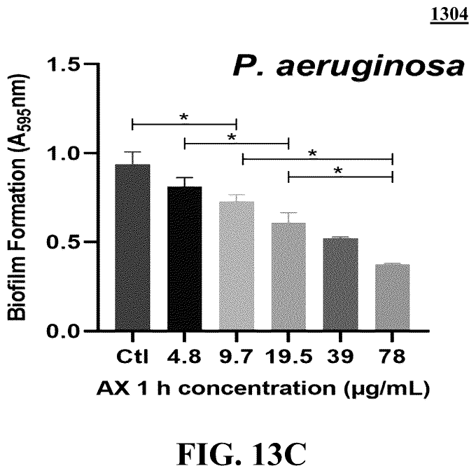

In this experiment, the dose-dependent potential of AX 1 h to inhibit biofilm formation was examined. FIG. 13A illustrates a graph 1300 for antibiofilm activity of exemplary multifunctional copolymeric nanoparticles synthesized with a reaction time of 1 hour (AX 1 h) on Enterococcus, consistent with one or more exemplary embodiments of the present disclosure. Referring to FIG. 13A, graph 1300 illustrates that the exemplary multifunctional copolymeric nanoparticles synthesized with a reaction time of 1 hour (AX 1 h) with a concentration of 39 μg/mL exhibited about 80% inhibitory effect on the biofilm formation of Enterococcus. Also, at the lower values than MIC (9.7 μg/mL), approximately 44% of the biofilm formation ability has been inhibited.

FIG. 13B illustrates a graph 1302 for antibiofilm activity of exemplary multifunctional copolymeric nanoparticles synthesized with a reaction time of 1 hour (AX 1 h) on Staphylococcus, consistent with one or more exemplary embodiments of the present disclosure. Referring to FIG. 13B, graph 1302 illustrates that results for Staphylococcus aureus show that 9.7 μg/mL and higher concentrations of the exemplary multifunctional copolymeric nanoparticles synthesized with a reaction time of 1 hour (AX 1 h) can decrease the biofilm strength of the bacterium from 100 to 17.74%. In addition to the significant antibacterial effect at MIC concentration (9.7 μg/mL), about 80% anti-biofilm effect is another advantage of the exemplary multifunctional copolymeric nanoparticles synthesized with a reaction time of 1 hour (AX 1 h).

FIG. 13C illustrates a graph 1304 for antibiofilm activity of exemplary multifunctional copolymeric nanoparticles synthesized with a reaction time of 1 hour (AX 1 h) on Pseudomonas aeruginosa, consistent with one or more exemplary embodiments of the present disclosure. Referring to FIG. 13C, evaluation of the antibiofilm effect of AX 1 h against P. aeruginosa showed an impressive reduction (about 45%) of biofilm formation at the MIC (19.5 μg/mL). Also, higher antibiofilm activity against P. aeruginosa can be achieved at higher concentrations of AX 1 h. Since P. aeruginosa is one of the most potent strains in biofilm formation, the potential to prevent biofilm development by such polymers makes them attractive antibiofilm agents.

Exemplary multifunctional copolymeric nanoparticles not only displayed good to excellent antibacterial activity with minimum inhibitory concentration (MIC) and minimum bactericidal concentration (MBC) value against Staphylococcus aureus, Enterococcus, Pseudomonas aeruginosa, and Escherichia coli but also demonstrated a substantial effect on reducing the number of biofilms even at low concentrations.

### Example 7: In-Vivo Antibacterial and Anti-Inflammatory Effect of Exemplary Multifunctional Polymeric Nanoparticles

In this example, in-vivo antibacterial and anti-inflammatory effects of exemplary multifunctional copolymeric nanoparticles synthesized with a reaction time of 1 hour (AX 1 h) were examined. In-vivo antibacterial activity of AX 1 h against P. aeruginosa and S. aureus was determined by studying contaminated burn wounds and quantifying the existing colony-forming units (CFUs) of bacteria in tissue on days 5 and 10.

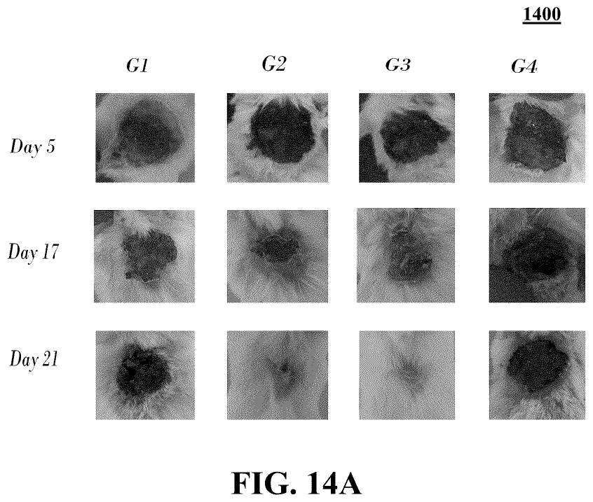

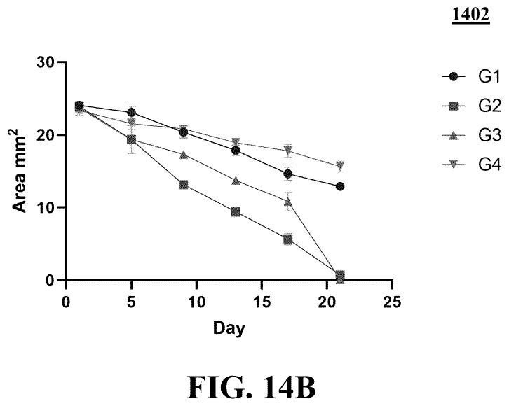

FIG. 14A illustrates an image 1400 of wound healing from days 5 to 21 in local treatment groups, including G2 (P. aeruginosa+AX 1 h) and G3 (S. aureus+AX 1 h) compared with untreated groups as controls, including G1 (P. aeruginosa infection and G4 (S. aureus infection) on mice, consistent with one or more exemplary embodiments of the present disclosure. FIG. 14B illustrates a plot 1402 wound area values from days 5 to 21 in local treatment groups, including G2 (P. aeruginosa+AX 1 h) and G3 (S. aureus+AX 1 h) compared with untreated groups as controls, including G1 (P. aeruginosa infection) and G4 (S. aureus infection) on mice, consistent with one or more exemplary embodiments of the present disclosure.

Referring to FIGS. 14A-14B, the full burn diameter contraction (G2 and G3) was achieved at the end of the treatment (day 21) as compared to the untreated ones (G1 and G4). Faster healing and significant shrinkage of the burn site are seen in the G2 and G3 groups treated with AX 1 h than in the control samples, and the rapid wound healing mechanism in the treated groups is probably due to induced angiogenesis, higher collagen deposition, and cell regeneration.

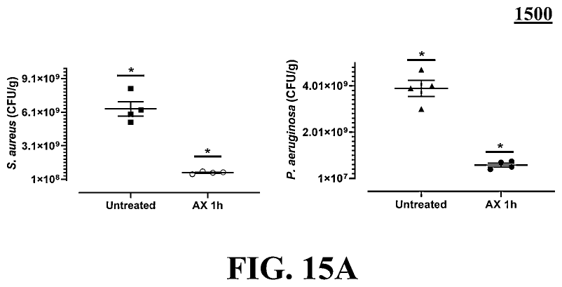

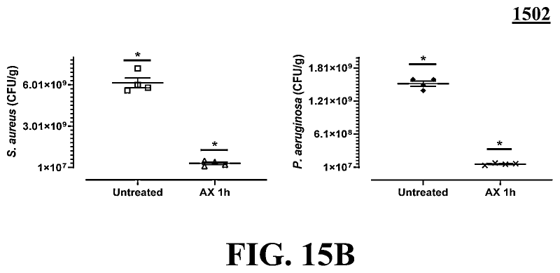

FIG. 15A illustrates a plot 1500 for bacterial load of burn wounds on day 5 in mice intradermally infected with P. aeruginosa and S. aureus (n=4 wounds per group), consistent with one or more exemplary embodiments of the present disclosure. FIG. 15B illustrates a plot 1502 for bacterial load of burn wounds on day 10 in mice intradermally infected with P. aeruginosa and S. aureus (n=4 wounds per group), consistent with one or more exemplary embodiments of the present disclosure. Referring to FIGS. 15A-15B, screening throughout all areas has continuously demonstrated decreases in bacterial burden. However, the wounds treated with AX 1 h showed a substantial decrease in bacterial counts than the control community (infected, untreated control) (P≤0.05).

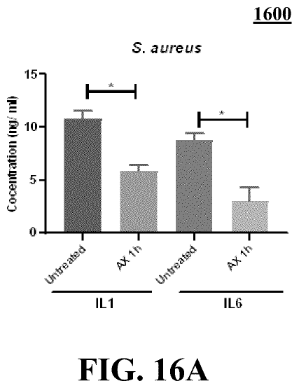

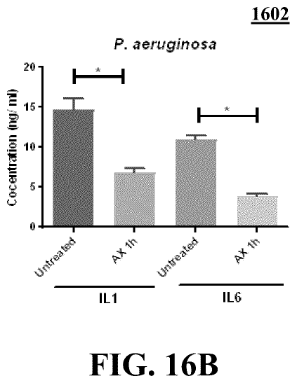

In addition to the antibacterial assays, the anti-inflammatory characteristics of the AX 1 h have been evaluated. Inflammatory elements of the mice's blood were studied by determining interleukin 1 (IL-1) and interleukin 6 (IL-6) concentrations in the groups treated with AX 1 h and untreated ones. FIG. 16A illustrates a plot 1600 for plasma levels of interleukin 1 (IL-1) and interleukin 6 (IL-6) after 21 days of wound infection with S. aureus in groups treated with exemplary multifunctional copolymeric nanoparticles synthesized with a reaction time of 1 hour (AX 1 h) and untreated groups, consistent with one or more exemplary embodiments of the present disclosure. (n=12-15 per group). FIG. 16B illustrates a plot 1602 for plasma levels of interleukin 1 (IL-1) and interleukin 6 (IL-6) after 21 days of wound infection with P. aeruginosa in groups treated with exemplary multifunctional copolymeric nanoparticles synthesized with a reaction time of 1 hour (AX 1 h) and untreated groups, consistent with one or more exemplary embodiments of the present disclosure. (n=12-15 per group). Error bars indicate SD, *p≤0.05

Referring to FIG. 16, plots 1600-1602 illustrate IL-1 and IL-6 levels in the groups treated with AX 1 h were less than half of the untreated mice. This decrease in the interleukin's level is significantly justifiable and indicates the high anti-inflammatory effect of exemplary multifunctional copolymeric nanoparticles against infectious wounds caused by P. aeruginosa and S. aureus.

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such away. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second, and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure and is not to be interpreted as reflecting an intention that the claimed implementations require more features than are expressly recited in each claim. Rather, as the following claims reflect, the inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in the light of the attached claims and their equivalents. Also, various modifications and changes may be made within the scope of the attached claims.

What is claimed is:

1. A multifunctional copolymeric nanoparticle, comprising:
a plurality of 1,4-diazabicyclo[2.2.2]octane (DABCO) monomers crosslinked with a plurality of bromoacetyl cystamine (BBAC) monomers, wherein each of the plurality of DABCO monomers and each of the plurality of BBAC monomers alternate in sequence, wherein the multifunctional copolymeric nanoparticle has a formula: 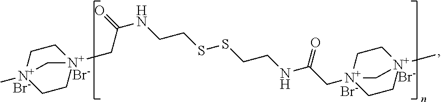
wherein n is an integer between 5 and 40.

2. The multifunctional copolymeric nanoparticle of claim 1, wherein the multifunctional copolymeric nanoparticle is cationic.

3. The multifunctional copolymeric nanoparticle of claim 1, wherein the multifunctional copolymeric nanoparticle has a functional group, the functional group comprising an ammonium group, an amide group, and a disulfide group.

4. The multifunctional copolymeric nanoparticle of claim 1, wherein the multifunctional copolymeric nanoparticle has a particle size between 1 nm and 2 μm.

5. The multifunctional copolymeric nanoparticle of claim 1, wherein the multifunctional copolymeric nanoparticle has antibacterial activity and anti-biofilm activity.

6. The multifunctional copolymeric nanoparticle of claim 5, wherein the multifunctional copolymeric nanoparticle has the antibacterial activity against gram-positive bacteria and gram-negative bacteria, the gram-positive bacteria comprising Staphylococcus aureus, Enterococcus, and the gram-negative bacteria comprising Pseudomonas aeruginosa and Escherichia coli.

7. The multifunctional copolymeric nanoparticle of claim 1, wherein the multifunctional copolymeric nanoparticle is a redox-sensitive copolymeric nanoparticle.

8. The multifunctional copolymeric nanoparticle of claim 1, wherein the multifunctional copolymeric nanoparticle is a transfection reagent for delivering a nucleic acid molecule into cells.

9. The multifunctional copolymeric nanoparticle of claim 8, wherein the nucleic acid molecule comprises at least one of deoxyribonucleic acid (DNA) and ribonucleic acid (RNA).

10. The multifunctional copolymeric nanoparticle of claim 9, wherein the nucleic acid molecule comprises at least one of a plasmid, siRNA, miRNA, and mRNA.

11. The multifunctional copolymeric nanoparticle of claim 8, wherein a ratio of the nucleic acid molecule to the multifunctional copolymeric nanoparticle is between 1:1 and 1:200.

12. A method for synthesizing a multifunctional copolymeric nanoparticle, the method comprising:
forming a mixture by mixing bromoacetyl cystamine (BBAC) and 1,4-diazabicyclo[2.2.2]octane (DABCO) with an alcoholic solvent at an equimolar ratio of the BBAC to the DABCO;
forming a precipitate by removing the alcoholic solvent from the mixture; and
obtaining a purified multifunctional copolymeric nanoparticle by purifying the precipitate through washing the precipitate with an organic solvent.

13. The method of claim 12, wherein mixing the BBAC and the DABCO with the alcoholic solvent comprises mixing the BBAC and the DABCO with the alcoholic solvent at a temperature between room temperature and 65° C. for a time period between 0.5 hour and 72 hours

14. The method of claim 12, wherein mixing the BBAC and the DABCO with the alcoholic solvent comprises mixing the BBAC with a concentration between 0.01 mM and 5 mM and the DABCO with a concentration between 0.01 mM and 5 mM with the alcoholic solvent.

15. The method of claim 12, wherein mixing the BBAC and the DABCO with the alcoholic solvent comprises stirring the BBAC and the DABCO with the alcoholic solvent using a stirrer.

16. The method of claim 12, wherein mixing the BBAC and the DABCO with the alcoholic solvent comprises mixing the BBAC and the DABCO with at least one of methanol, ethanol, and isopropanol.

17. The method of claim 12, wherein removing the alcoholic solvent from the mixture comprises removing the alcoholic solvent from the mixture using a rotary evaporator.

18. The method of claim 12, wherein washing the precipitate with the organic solvent comprises washing the precipitate with at least one of ethyl acetate, acetone, chloroform, and diethyl ether.

19. The method of claim 12 further comprising forming a homogenous multifunctional copolymeric nanoparticle by filtering the purified multifunctional copolymeric nanoparticle using a syringe filter with a pore size of 220 nm.
